(19) United States
(12) Patent Application Publication
(10) Pub. No.: US 2015/0119747 A1
(43) Pub. Date: Apr. 30, 2015

(54) System and Method for Acquisition of Biopotential Signals With Electrode-Tissue Impedance Measurement

(71) Applicant: IMEC VZW, Leuven (BE)

(72) Inventors: Tom Torfs, Kraainem (BE); Refet Firat Yazicioglu, Leuven (BE)

(73) Assignee: IMEC VZW, Leuven (BE)

(21) Appl. No.: 14/528,931

(22) Filed: Oct. 30, 2014

(30) Foreign Application Priority Data
Oct. 31, 2013 (EP) 13191015.0

(51) Int. Cl. A61B 5/053; A61B 5/00

(52) U.S. Cl. CPC A61B 5/053; A61B 5/7278
USPC 600/547

(57) ABSTRACT

A system for the acquisition of biopotential signals, comprising at least a first electrode configured for detecting a biopotential signal within a signal bandwidth of interest and being connected to an impedance detection module that provides a first electrode voltage. The impedance detection module comprises a current generation circuit connected in parallel to an amplifier. The current generation circuit comprises an AC current generator configured to generate a first current signal through the first electrode. The first current signal has a frequency outside of the signal bandwidth of interest. The current generation circuit also comprising a capacitor connected between the input of the amplifier and the AC current generator so as to isolate the AC current generator from the amplifier input at the signal bandwidth of interest. The system also including a signal processor configured to calculate a component value of a first and a second electrode-tissue impedance based on a difference between the first electrode voltage and a second electrode voltage.

### CROSS-REFERENCE TO RELATED APPLICATION

This application claims priority to European Patent Application No. 13191015.0 filed on Oct. 31, 2013, the contents of which are hereby incorporated by reference.

### TECHNICAL FIELD

The present description relates generally to the field of biopotential signal acquisition systems and more specifically to a system and a method in the art with electrode-tissue impedance measurement capabilities.

### BACKGROUND

Ambulatory monitoring of biopotential signals (ECG, EEG, EMG, etc.) is a highly relevant topic in personal healthcare. A key technical challenge in such application environments is overcoming motion artifacts that significantly affect the recorded biopotential signals. A possible approach to tackle this problem is to collect data from other sensors that have maximum correlation with the motion artifact signal and minimal correlation with the biopotential signals. Some known systems measure the electrode-tissue impedance, which is then used as a reference signal input for removing such motion artifacts in the biopotential signal.

One known electrode-skin impedance monitoring system is disclosed in an article titled “A 2.4 μA continuous-time electrode-skin impedance measurement circuit for motion artifact monitoring in ECG acquisition systems,” by Sunyoung Kim et al., VLSI Circuits (VLSIC), 2010, pp. 219-220, 16-18 Jun. 2010. The electrode-tissue impedance is measured by applying an alternating current (AC) of constant amplitude and frequency into the electrodes and detecting the resulting differential voltage between the electrodes, through the voltage amplifier that also measures the ECG signal.

Another known technique for measuring the electrode-tissue impedance is disclosed in an article titled “Correlation Between Electrode-Tissue Impedance and Motion Artifact in Biopotential Recordings,” by Dilpreet Buxi et al., IEEE Sensors Journal, Vol. 12, no. 12, December 2012, pp. 3373-3383. In this system the phase of a single current source can be selected between 0° and 180° in mode D and mode T, respectively. Mode D leads to the generation of a common-mode stimulation current that can be used to monitor the impedance difference between two lead electrodes. Mode T leads to the generation of a differential stimulation current that can be used to measure the sum of impedances of the lead electrodes or the total impedance.

However, state of the art systems are not well suited for biopotential signal acquisition systems in ambulatory environments where the sensors/electrodes are less tightly strapped to the body and/or no gel is used and therefore some degree of relative sensor to body motion will occur.

### SUMMARY

According to one aspect of the present disclosure, a new system and method for acquisition of biopotential signals is provided.

According to an exemplary embodiment of the present description a system for the acquisition of biopotential signals includes a first electrode configured for detecting a biopotential signal within a signal bandwidth of interest and being connected to an impedance detection module which provides a first electrode voltage. The impedance detection module includes a current generation circuit with an AC current generator configured to generate a first current signal through the first electrode. The first current signal may have a frequency outside of the signal bandwidth of interest. In this example, the system also includes an amplifier connected in parallel to the current generation circuit, and signal processing means for calculating a component value of at least a first electrode-tissue impedance based on the difference between the first electrode voltage and a second electrode voltage. The current generation circuit comprises a capacitor connected between the input of the amplifier and the AC current generator so as to isolate the AC current generator from the amplifier input at the signal bandwidth of interest.

According to an exemplary embodiment, the first electrode impedance is greater than 1 megohm.

According to another exemplary embodiment, the signal bandwidth of interest is below 250 hertz.

According to another exemplary embodiment, the first current signal has a frequency greater than 1 kilohertz.

According to another exemplary embodiment, the value of the current generation circuit capacitor is designed such as to reduce the amplifier's input impedance by less than 25%.

According to another exemplary embodiment, the value of the current generation circuit capacitor is designed such as to obtain values of the current generation circuit impedance greater than 10 gigaohm at the signal bandwidth of interest.

According to another exemplary embodiment, the value of the current generation circuit capacitor is between 0.1 to 20 picofarad.

According to another exemplary embodiment, the AC current generator is designed so as to have an output impedance which is at least 5 times higher than the impedance of the capacitor at the frequency of the first current signal.

According to another exemplary embodiment, the system for acquisition of biopotential signals further includes a second electrode configured for detecting a biopotential signal within a signal bandwidth of interest and being connected to an impedance detection module which provides a second electrode voltage. In this embodiment, the impedance detection module comprises a current generation circuit with an AC current generator configured to generate a second current signal through the second electrode. Further, in one example, the first current signal (IS1) and the second current signal are 180 degrees out of phase.

According to another exemplary embodiment, the system for acquisition of biopotential signals further comprises a second electrode configured for detecting a biopotential signal within a signal bandwidth of interest and being connected to an impedance detection module which provides a second electrode voltage. In this embodiment, the impedance detection module comprises a current generation circuit with an AC current generator configured to generate a second current signal through the second electrode, a bias electrode configured for biasing the subject's body, and is so arranged that when the first current signal and the second current signal are in phase, the net resulting current flows into the bias electrode.

According to another exemplary embodiment, at least one of the electrodes is a non-contact or a dry contact electrode. The non-contact electrode may be a non-contact capacitive electrode.

According to an exemplary embodiment, the biopotential signal is an ECG, an EEG, or and EMG biopotential signal.

According to another aspect of the present description, a method for the acquisition of biopotential signals comprises detecting a biopotential signal within a signal bandwidth of interest with a first electrode and generating a first current signal through the first electrode. In this example, the first current signal has a frequency outside of the signal bandwidth of interest. The method also includes isolating the first current signal from the detected biopotential signal at the signal bandwidth of interest, generating a first and a second electrode voltage, and calculating a component value of at least a first electrode-tissue impedance based on the difference between the first electrode voltage and a second electrode voltage.

Certain potential objects and advantages of various new and inventive aspects have been described above. It is to be understood that not necessarily all such objects or advantages may be achieved in accordance with any particular embodiment of the present disclosure. Those skilled in the art will recognize that the solution of the present disclosure may be embodied or carried out in a manner that achieves or optimizes one advantage or group of advantages without necessarily achieving other objects or advantages.

### BRIEF DESCRIPTION OF THE DRAWINGS

The above and other aspects of the system and method for acquisition of biopotential signals according to the present disclosure will be shown and explained with reference to the non-restrictive example embodiment(s) described hereinafter.

FIGS. 1A and 1B show a first exemplary block diagram of a system for acquisition of biopotential signals according to an embodiment.

FIGS. 2A and 2B show a second exemplary block diagram of a system for acquisition of biopotential signals according to an embodiment.

FIG. 3 shows a third exemplary block diagram of a system for acquisition of biopotential signals according to an embodiment.

FIG. 4 shows a fourth exemplary block diagram of a system for acquisition of biopotential signals according to an embodiment.

FIGS. 5A and 5B show a fifth exemplary block diagram of a system for acquisition of biopotential signals according to an embodiment.

FIG. 6 shows a sixth exemplary block diagram of a system for acquisition of biopotential signals according to an embodiment.

### DETAILED DESCRIPTION

In the following description of exemplary embodiments, various features may be grouped together in a single embodiment, figure, or description thereof for the purpose of streamlining the disclosure and aiding in the understanding of one or more of the various inventive aspects. This is however not to be interpreted as the various embodiments requiring more features than the ones expressly recited in the claims. Furthermore, combinations of features of different embodiments are meant to be within the scope of the disclosure, as would be clearly understood by those skilled in the art. Additionally, in other instances, well-known methods, structures and techniques have not been shown in detail in order not to obscure an understanding of the description. Further, it should be understood that the word “exemplary” is used herein to mean “serving as an example, instance, or illustration.” Any embodiment or feature described herein as “exemplary” is not necessarily to be construed as preferred or advantageous over other embodiments or features.

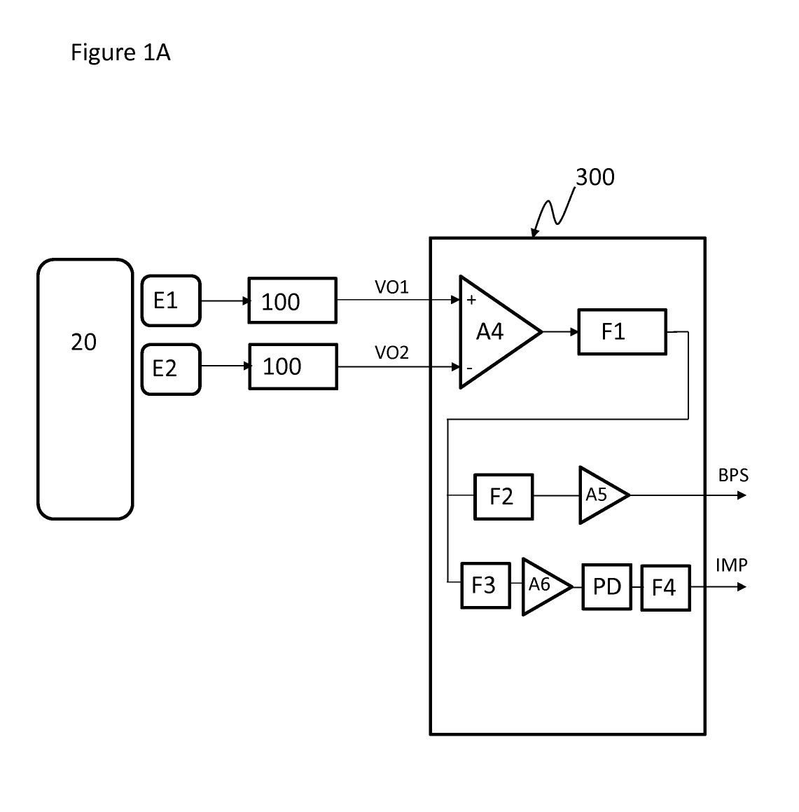

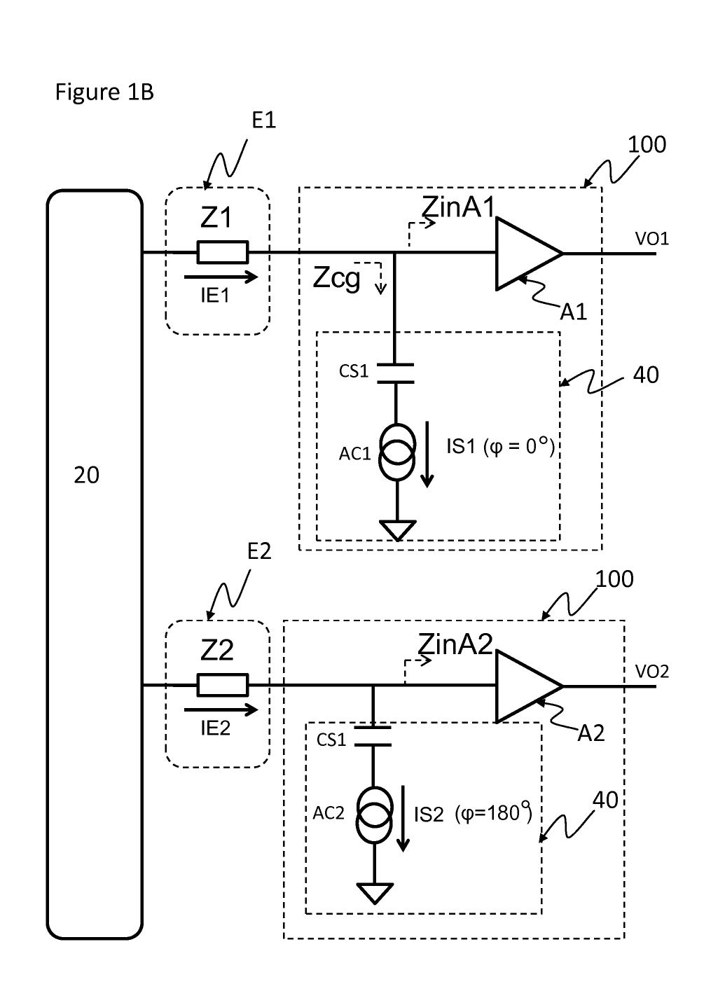

FIG. 1A shows a first exemplary block diagram of a system for acquisition of biopotential signals according to an embodiment. The figure illustrates two electrodes E1, E2 that are applied to a human body 20, for example, by being attached to or placed in close proximity to the subject's skin or tissue. Each electrode is connected to an impedance detection module 100 that provides an electrode voltage signal VO1, VO2. The figure also illustrates schematically a signal processing unit 300, which basically extracts or calculates, from the first and second voltage electrode signals VO1, VO2, a biopotential signal BPS and an electrode-tissue impedance signal IMP. The signal processing unit 300 may comprise a differential amplifier A4 and further signal processing means or components, which extract the biopotential and electrode-tissue impedance signals, which are multiplexed on the same signal but at different frequencies at the output of the differential amplifier A4. The signal processing means may comprise, for example, an optional mains notch F1, and in the biopotential signal path, a low-pass filter F2 and a gain amplifier A5, and in the electrode-tissue impedance signal path, a band-pass filter F3, a gain amplifier A6, a peak detector PD and a low-pass filter F4.

It is understood that the system may comprise further signal processing means or digital processing units or circuits that may use the electrode-tissue impedance signal IMP for motion artifact reduction purposes, for detecting whether and how well an electrode is making contact to the body 20, and/or for assessing the quality and variation over time of the electrode connection and, for example, assigning a suitable confidence level to the obtained signals or features extracted out of them or discarding fragments of signals entirely.

FIG. 1B shows a more detailed representation of some aspects of the system of FIG. 1A, and more specifically, the electrodes E1, E2 and the impedance detection module 100. The electrodes E1, E2 are represented by an electrode-tissue impedance Z1, Z2 which is connected to a current generation circuit 40 and to an amplifier A1, A2. The current generation circuit 40 comprises an AC current generator AC1, AC2 and a capacitor CS1 connected in series. Each AC current generator AC1, AC2 generates a current signal IS1, IS2, IE1, 1E2 through the electrodes which has a frequency outside of the signal bandwidth of interest of the biopotential signal. According to an exemplary embodiment, the frequency of the generated current signal IS1, IS2 is higher than 1 kHz.

According to an exemplary embodiment of the disclosure, the amplifier A1 is a high input impedance amplifier. The input impedance Zcg of the current generation circuit 40 is also high compared to the input impedance of the biopotential amplifier A1 so that the total input impedance ZinA1, ZinA2 is not severely degraded. According to an exemplary embodiment, the AC current source AC1, AC2 with the capacitor CS1 in series allows maintaining a very large input impedance at signal frequencies of the biopotential signal of interest. According to an exemplary embodiment, the biopotential signal frequencies of interest are below 100 or 250 Hz. According to another exemplary embodiment, the value of the capacitor CS1 lies in the picofarad range, for example, 1 pF. The AC current source may be designed with relaxed output impedance requirements, for example, an output impedance below 1 GΩ.

Also according to an exemplary embodiment of the disclosure, the capacitor CS1 advantageously separates or isolates the current source IS1, IS2 from the biopotential signal at the input of the amplifier A1, A2. According to an exemplary embodiment, the series capacitor effectively isolates the current source at biopotential signal frequencies, resulting in an additional input load impedance of 16 GΩ, which will reduce the total input impedance ZinA1, ZinA2 by only 25%.

Further, according to an exemplary embodiment, the AC current source AC1, AC2 has an output impedance that is significantly higher than the series impedance of the coupling capacitor CS1 but only at the frequency of the generated current IS1, IS2. For example, in case the frequency of the generated current is 10 kHz, an AC current source AC1, AC2 with an output impedance higher than 16 MΩ suffices to function as a good AC current source. According to an exemplary embodiment, the AC current source AC1, AC2 is implemented as a Howland current pump. A Howland current pump can advantageously produce a voltage-controlled output current, simplifying the generation of sinusoidal or arbitrary current shapes, for example, through a DAC. Additionally, a single stage can both source and sink current as required. For small currents, such as in the nA range, a high output impedance can be achieved without high-precision or trimmed resistors.

According to an embodiment of the disclosure, the impedance detection module 100 applies an AC current IS1, IS2 of constant amplitude and frequency into the electrodes E1, E2 and the resulting differential voltage between the electrodes VO1, VO2 is measured through the voltage differential amplifier A4 (as shown in FIG. 1A). When the current in the electrodes is 180° out of phase, the electrode current IE1, IE2 will flow from one electrode to the other, and the resulting differential voltage signal at the output of the differential amplifier A4 will be proportional to the sum of the electrode-tissue impedances Z1, Z2, since the body impedance is negligible. According to an exemplary embodiment, the electrode-tissue impedances Z1, Z2, are greater than 1 MΩ. According to another exemplary embodiment, the electrodes E1 and E2 are non-contact electrodes. It is understood that, according to exemplary embodiments, the disclosure may also be advantageous for implementations in which the electrodes E1 and E2 are dry-contact electrodes.

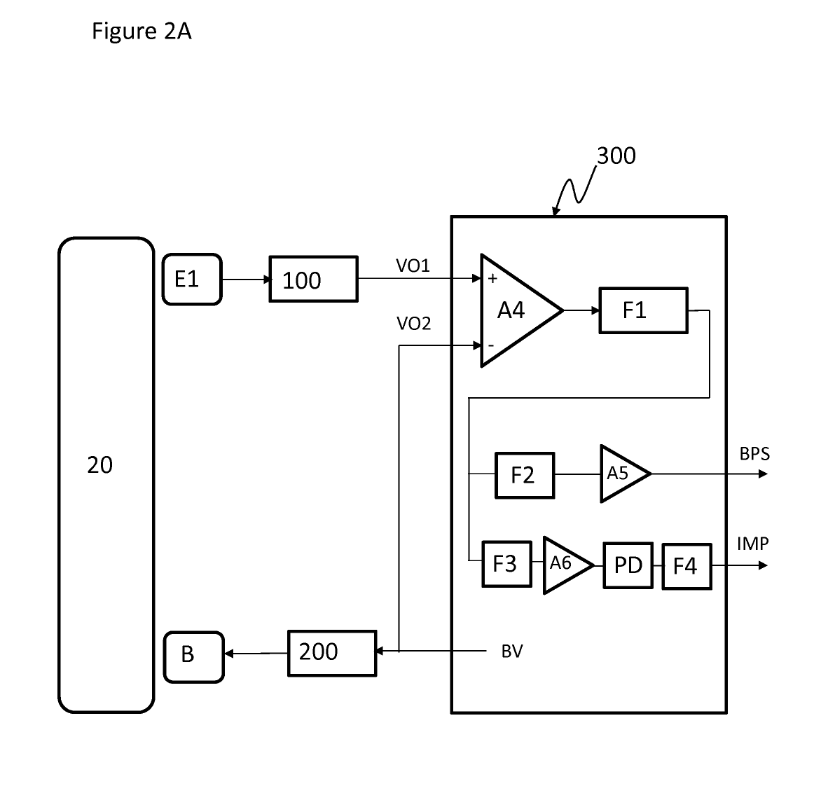

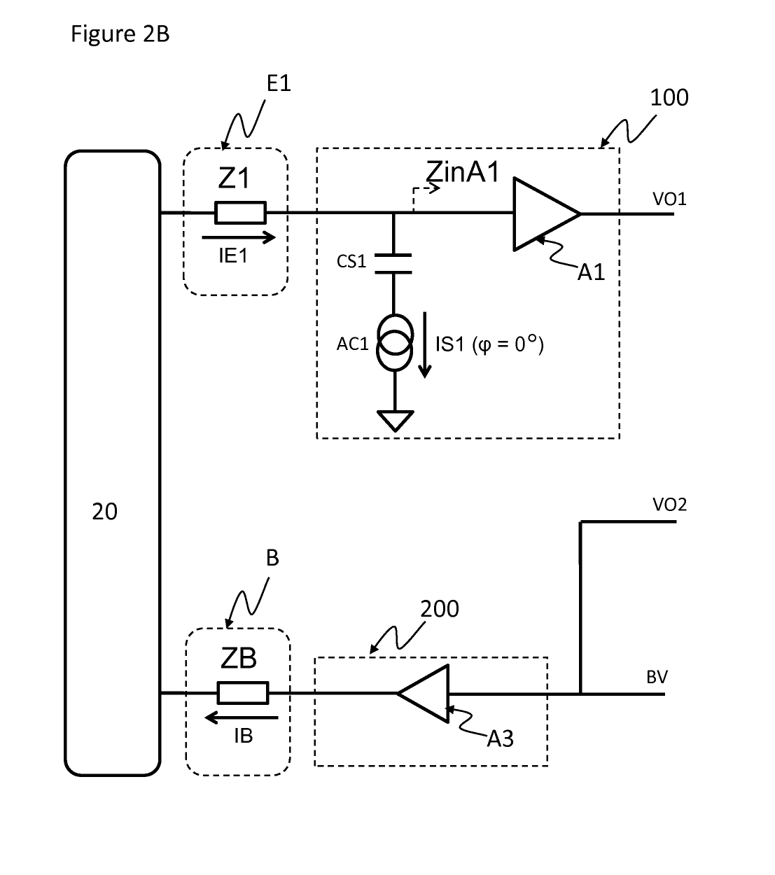

FIG. 2A shows a second exemplary block diagram of a system for acquisition of biopotential signals according to an embodiment. The figure illustrates two electrodes E1, B that are applied to a human body 20. In this example, the second electrode B is a bias electrode connected to a biasing circuit 200 and to the signal processing unit 300 that generates the biasing voltage BV. It is understood that in other possible exemplary embodiments, the first electrode voltage VO1 and the second electrode voltage VO2 can be interchanged at the input of the differential amplifier A4.

FIG. 2B shows a more detailed representation of some aspects of the system of FIG. 2A, and more specifically, the electrodes E1, B, the impedance detection module 100, and the biasing circuit 200. Each of the electrodes E1, B is represented by an electrode-tissue impedance Z1, ZB, respectively. In case of the first electrode E1, the electrode-tissue impedance Z1 is connected to an impedance detection module 100, such as as described in relation to FIG. 1B, and in case of the second electrode B, the electrode-tissue impedance ZB is connected to the biasing circuit 200 comprising an amplifier A3. According to an embodiment of the disclosure, the bias electrode B has a low electrode-tissue impedance ZB compared to the first electrode's electrode-tissue impedance Z1, e.g., greater than 1 MΩ, and the resulting differential voltage signal at the output of the differential amplifier A4 will be proportional to the electrode-tissue impedance of the first electrode Z1, since the body impedance is negligible. According to another exemplary embodiment, the first electrode E1 may be a non-contact or a dry-contact electrode.

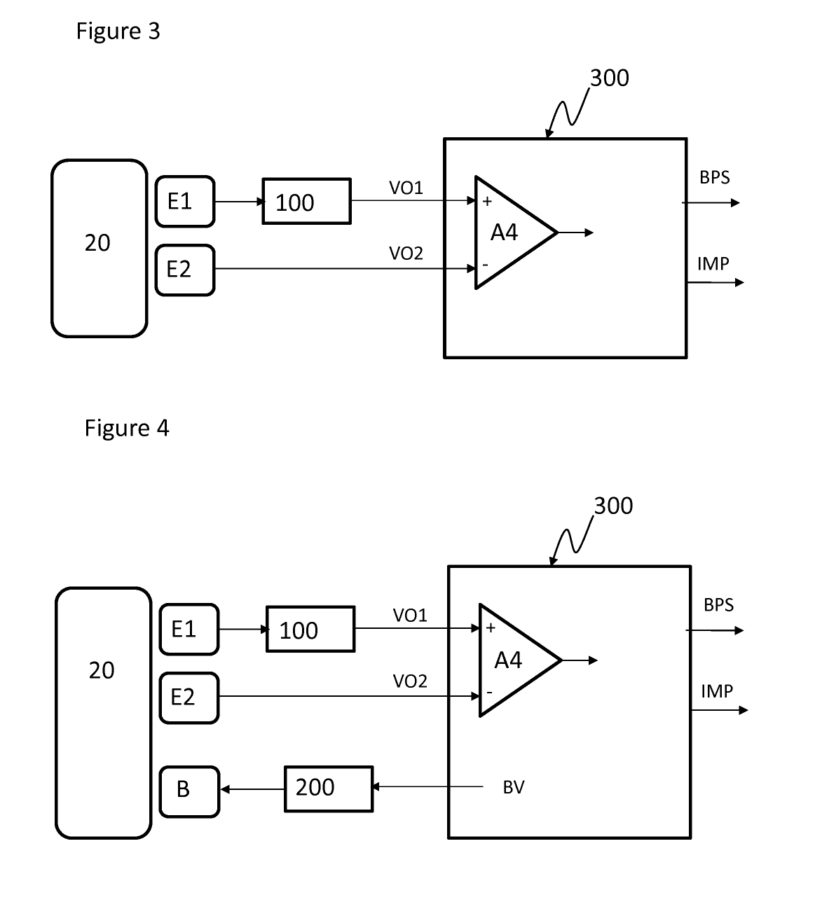

FIG. 3 shows a third exemplary block diagram of a system for acquisition of biopotential signals according to an embodiment. The figure illustrates two electrodes E1, E2 that are applied to a human body 20. In this example, the second electrode E2 is a low impedance electrode, e.g., a contact electrode, which generates a second electrode voltage VO2. The first electrode E1 is connected to the impedance detection module 100 as described in relation to FIG. 1B, and provides a first electrode voltage signal VO1. It is also understood that in other possible exemplary embodiments, the first electrode and the second electrode voltage signals VO1, VO2 can be interchanged at the input of the differential amplifier A4. According to another exemplary embodiment, the first electrode E1 is a non-contact or a dry-contact electrode. According to an embodiment, the first electrode E1 has an electrode-tissue impedance Z1 greater than 1 MΩ. According to an embodiment of the disclosure, the second electrode E2 has low electrode-tissue impedance Z2 compared to the first electrode's electrode-tissue impedance Z1, and the resulting differential voltage signal at the output of the differential amplifier A4 will be proportional to the electrode-tissue impedance of the first electrode Z1, since the body impedance is negligible.

FIG. 4 shows a fourth exemplary block diagram of a system for acquisition of biopotential signals according to an embodiment. The figure illustrates three electrodes E1, E2, B that are applied to a human body 20. In this example, the second electrode E2 is a low electrode-tissue impedance electrode, e.g., a contact electrode, which generates a second electrode voltage VO2, and the third electrode B is a bias electrode connected to a biasing circuit 200, such as disclosed in relation to FIG. 2B, and to the signal processing unit 300 that generates the biasing voltage BV. The first electrode E1 is connected to the impedance detection module 100 as described in relation to FIG. 1B and provides a first electrode voltage signal VO1. It is also understood that in other possible exemplary embodiments, the first electrode and the second electrode voltage signals VO1, VO2 can be interchanged at the input of the differential amplifier A4. According to another exemplary embodiment, the first electrode E1 is a non-contact electrode. According to an exemplary embodiment, the first electrode E1 has an electrode-tissue impedance Z1 greater than 1 MΩ. According to an exemplary embodiment, this configuration works as the one disclosed in FIG. 3 but with improved rejection of common mode noise or CMRR. According to exemplary embodiments the disclosure, this configuration is also advantageous for applications with dry-contact electrodes.

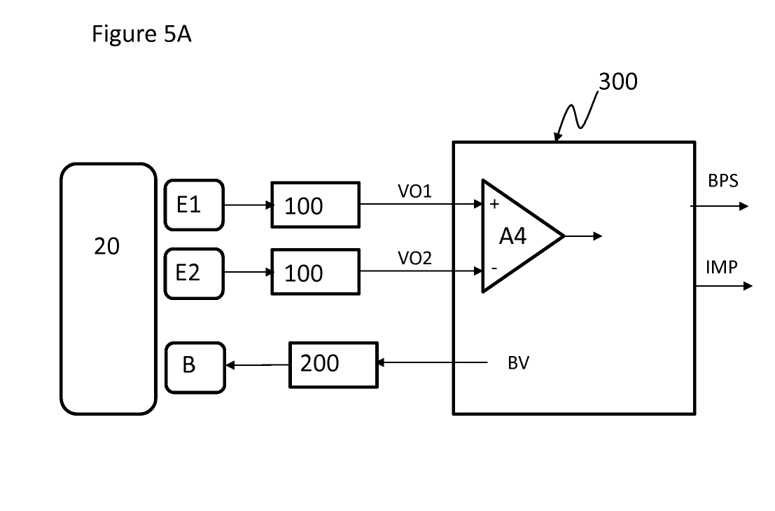

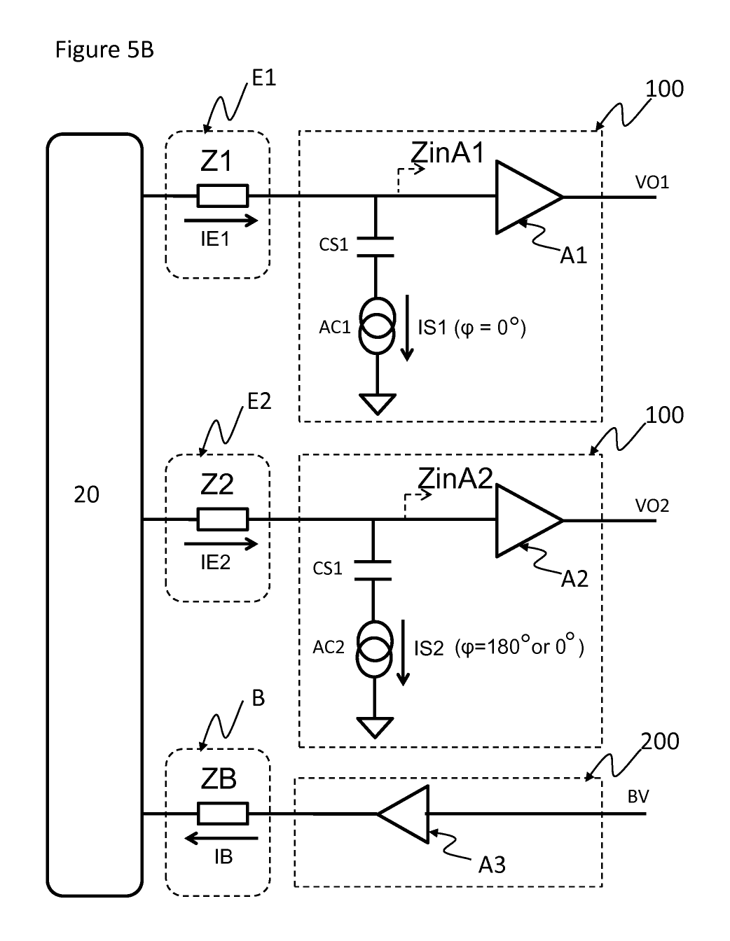

FIG. 5A shows a fifth exemplary block diagram of a system for acquisition of biopotential signals according to an embodiment. The figure illustrates three electrodes E1, E2, B that are applied to a human body 20. In this example, the first and the second electrodes E1, E2 are connected to an impedance detection module 100 as described in relation to FIG. 1B, and are configured to generate a first and a second electrode voltage VO1, VO2, respectively. The third electrode B is a bias electrode connected to a biasing circuit 200, as described in relation to FIG. 2B, and to the signal processing unit 300 that generates the biasing voltage BV. According to an exemplary embodiment, the first and second electrodes E1, E2 are non-contact electrodes. According to an exemplary embodiment, the first and second electrodes E1, E2 have an electrode-tissue impedance Z1, Z2 greater than 1 MΩ. The bias electrode B may be a low electrode-tissue impedance electrode, e.g., a contact electrode. According to exemplary embodiments the disclosure, this configuration is also advantageous for applications in which the first and/or the second electrode E1, E2 is a dry-contact electrode.

FIG. 5B shows a more detailed representation of some aspects of the system of FIG. 5A, and more specifically, the electrodes E1, E2, B, the impedance detection module 100 and the biasing circuit 200. Each of the electrodes E1, E2, B is represented by an electrode-tissue impedance Z1, Z2, ZB. In the present example, the electrode-tissue impedances Z1, Z2 of the first and second electrodes E1, E2 are connected to an impedance detection module 100 as described in relation to FIG. 1B, and the electrode-tissue impedance ZB of the bias electrode B is connected to the biasing circuit 200 comprising an amplifier A3.

According to an embodiment of the disclosure, the impedance detection module 100 applies an AC current IS1, IS2 of constant amplitude and frequency into the electrodes E1, E2 and the resulting differential voltage between the electrodes VO1, VO2 is measured through the voltage differential amplifier A4 (as shown in FIG. 1A). When the current in the electrodes is 180° out of phase, the electrode current IE1, IE2 will flow from one electrode to the other, and the resulting differential voltage signal at the output of the differential amplifier A4 will be proportional to the sum of the electrode-tissue impedances Z1, Z2, since the body impedance is negligible. When the current in both electrodes IE1, IE2 is in phase, the net resulting current will flow into the bias electrode B, and the resulting differential voltage signal at the output of the differential amplifier A4 will be proportional to the difference of the of the electrode-tissue impedances Z1, Z2, since the body impedance is negligible.

It is understood that without parasitics, the current through the electrodes IE1, IE2 equals the generated AC current IS1, IS2 in the current generation circuit 40.

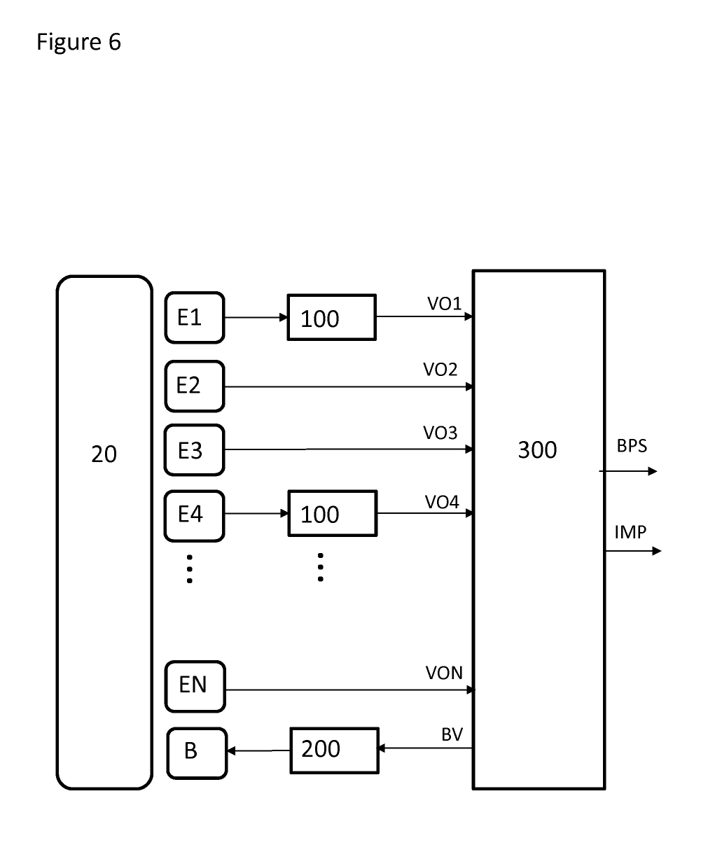

FIG. 6 shows a sixth exemplary block diagram of a system for acquisition of biopotential signals according to an embodiment. The figure illustrates N+1 electrodes E1 to EN, B that are applied to a human body 20, wherein some of the electrodes, for example, E1 and E4, are connected to an impedance detection module 100 as described in relation to FIG. 1B, and some of the electrodes, for example, E2, E3, EN, are not. A bias electrode B is also present, and is connected to a biasing circuit 200, as described in relation to FIG. 2B, and to the signal processing unit 300 that generates the biasing voltage BV. According to an embodiment of the disclosure, the signal processing unit 300 may consider any pair of electrode voltages VO1 to VON, BV in order to calculate a component value of a first or a first and a second electrode-tissue impedance based on the difference between a first electrode voltage and a second electrode voltage, as is described in previous figures and embodiments. According to exemplary embodiments, the electrodes E1 and/or E4 may be non-contact and/or dry-contact electrodes.

1. A system for the acquisition of biopotential signals, comprising:
at least a first electrode configured for detecting a biopotential signal within a signal bandwidth of interest and being connected to an impedance detection module that provides a first electrode voltage;
the impedance detection module comprising a current generation circuit connected in parallel to an amplifier;
the current generation circuit comprising an AC current generator configured to generate a first current signal through the first electrode, wherein the first current signal has a frequency outside of the signal bandwidth of interest, and a capacitor connected between the input of the amplifier and the AC current generator so as to isolate the AC current generator from the amplifier input at the signal bandwidth of interest; and
a signal processor configured for calculating a component value of at least a first electrode-tissue impedance based on a difference between the first electrode voltage and a second electrode voltage.

2. The system for the acquisition of biopotential signals according to claim 1, wherein the first electrode-tissue impedance is greater than 1 megohm.

3. The system for the acquisition of biopotential signals according to claim 1, wherein the signal bandwidth of interest is below 250 hertz.

4. The system for the acquisition of biopotential signals according to claim 1, wherein the first current signal has a frequency greater than 1 kilohertz.

5. The system for the acquisition of biopotential signals according to claim 1, wherein the value of the current generation circuit capacitor is designed such as to reduce the amplifier's input impedance by less than 25%.

6. The system for the acquisition of biopotential signals according to claim 1, wherein the value of the current generation circuit capacitor is designed such as to obtain values of the current generation circuit impedance greater than 10 gigaohm at the signal bandwidth of interest.

7. The system for the acquisition of biopotential signals according to claim 1, wherein the value of the current generation circuit capacitor is in a range between 0.1 to 20 picofarad.

8. The system for the acquisition of biopotential signals according to claim 1, wherein the AC current generator is designed so as to have an output impedance that is at least 5 times higher than the impedance of the capacitor at the frequency of the first current signal.

9. The system for the acquisition of biopotential signals according to claim 1, wherein the AC current generator is implemented as a Howland current pump.

10. The system for the acquisition of biopotential signals according to claim 1, wherein the first electrode is a non-contact or a dry-contact electrode.

11. The system for the acquisition of biopotential signals according to claim 1, further comprising:
a second electrode configured for detecting a biopotential signal within a signal bandwidth of interest and being connected to a second impedance detection module that provides a second electrode voltage;
the second impedance detection module comprising a current generation circuit with an AC current generator configured to generate a second current signal through the second electrode; and
wherein the first current signal and the second current signal are 180 degrees out of phase.

12. The system for the acquisition of biopotential signals according to claim 1, further comprising:
a second electrode configured for detecting a biopotential signal within a signal bandwidth of interest and being connected to a second impedance detection module that provides a second electrode voltage;
the second impedance detection module comprising a current generation circuit with an AC current generator configured to generate a second current signal through the second electrode;
a bias electrode configured for biasing a subject's body; and
wherein when the first current signal and the second current signal are in phase, a net resulting current flows into the bias electrode.

13. A method for the acquisition of biopotential signals, comprising:
detecting a biopotential signal within a signal bandwidth of interest with at least a first electrode;
generating a first current signal through the first electrode, the first current signal having a frequency outside of the signal bandwidth of interest;
isolating the first current signal from the detected biopotential signal at the signal bandwidth of interest;
generating a first and a second electrode voltage; and
calculating a component value of at least a first electrode-tissue impedance based on a difference between the first electrode voltage and a second electrode voltage.
